(19) United States
(12) Patent Application Publication
(10) Pub. No.: US 2023/0131784 A1
(43) Pub. Date: Apr. 27, 2023

(54) ENDOSCOPIC ARTICULATION DEVICE

(71) Applicant: GYRUS ACMI, INC. D/B/A OLYMPUS SURGICAL TECHNOLOGIES AMERICA, Westborough, MA (US)

(72) Inventors: Spence Edward Cornia, Sandy, UT (US); Dustin Edward Gorringe, Riverton, UT (US); Justin Garth Tidwell, North Salt Lake, UT (US); Derek Alder Hutchison, Vineyard, UT (US); Blake Salgat, Park City, UT (US); Kelly David Christensen, Centerville, UT (US)

(21) Appl. No.: 18/046,801

(22) Filed: Oct. 14, 2022

Related U.S. Application Data
(60) Provisional application No. 63/270,913, filed on Oct. 22, 2021.
(60) Provisional application No. 63/267,578, filed on Feb. 4, 2022.

(51) Int. Cl. A61B 1/005

(52) U.S. Cl. CPC A61B 1/0052; A61B 1/0057; A61M 25/0136

(57) ABSTRACT

An articulating medical device including handle and an articulating elongate member extending from the handle. The handle is a housing which includes at least one rotatable member, defining an arcuate track extending for a partial rotation about a center axis of rotation, at least one clamp captured, translatable, and rotatable within the arcuate track, and at least one articulation cable, fastened to the at least one clamp and extending outward from the handle, toward the articulating elongate member, including extending, when under tension, along a first lateral periphery of the at least one rotatable member and within a channel formed in the rotatable member. The at least one clamp translates within the arcuate track to accommodate slack in the at least one articulation cable when the at least one articulation cable is not under tension.

### PRIORITY CLAIM

This application claims the benefit of priority to U.S. Provisional Patent Application No. 63/270,913, filed Oct. 22, 2021, and U.S. Provisional Patent Application No. 63/267,578, filed Feb. 4, 2022, the contents of which are incorporated herein by reference in their entireties.

### BACKGROUND

Endoscopic devices have an articulation component which allows the user to view, modify, repair, or otherwise interact with cavities and lumens within a human body. In some examples, endoscopes use articulation wires or cables to manipulate or control movement of an articulating insertion portion of an endoscope. For example, a pulley system having rotatable portions inside a handle or other control portion is used to guide, move or control the articulation wire or wires. For example, for a four-direction control there are two pulleys or rotatable portions, and respective components such as knobs as user-manipulated control mechanisms on the handle and wires within the handle. In such an example, a first cable articulates the insertion portion in a vertical direction and the second cable articulates the insertion portion in a horizontal direction. In other examples, the insertion portion is moved in one direction and the associated rotatable components only control one direction of movement, either horizontal or vertical.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1A illustrates an example of the interior hollow portion of an articulating endoscopic device.

FIG. 1B illustrates an example of the interior hollow portion of an articulating endoscopic device.

FIG. 2A illustrates an example of the interior of a hollow portion of an articulating endoscopic device with external rotatable control mechanisms.

FIG. 2B illustrates an exploded view of the interior of a hollow portion of an articulating endoscopic device with external rotatable control mechanisms.

FIG. 2C illustrates an exploded view of the interior of a hollow portion of an articulating endoscopic device with external rotatable control mechanisms.

FIG. 3A illustrates an example of a rotatable member with an arcuate track.

FIG. 3B illustrates an example of a top view of the interior hollow portion of an articulating endoscopic device with rotatable member and clamps within an arcuate track.

FIG. 3C illustrates an example of an exploded view of a rotatable member with an arcuate track.

FIG. 3D illustrates an example of a rotatable member.

FIG. 4 illustrates a clamp with a cable within a through hole in the clamp.

FIG. 5 illustrates top view of the interior hollow portion of an articulating endoscopic device with rotatable member and clamps within an arcuate track.

FIG. 6A-6E illustrate an example of the movement of rotatable member and the articulating cables.

FIG. 7 illustrate an example of a first rotatable member coupled with a second rotatable member.

### SUMMARY

An endoscopic device can use wires or cables to control articulation of insertion portions. These articulation wires or cable (hereinafter “cable”) can become disengaged with an associated rotatable member, articulation system or similar mechanism to move the cables. Therefore, a need has been identified to have a mechanism with cables of an articulation component are coupled to a rotatable member or pulley system which provides some managed freedom, or slack, of the cables. Cables in articulation devices, undergo tension or slack when the associated rotatable mechanisms move the cable. The rotation transfers movement to the insertion articulation portions of the endoscopic device. In an example, slack or tension in the cables translates a corresponding horizontal or vertical movement to the articulating member. Coupling components, for example, clamps, barrels, fasteners or similar parts (hereinafter clamps) couple the cables to the rotatable mechanisms. In an example, clamps travel within grooves, tracks or paths of the rotatable member, thereby causing the wires or cables to articulate from a position under tension to a slack position.

### DETAILED DESCRIPTION

This document describes, among other things, a device or mechanism to move an articulating feature in a medical device such as an endoscope or other medical device with an articulating component. The medical device associated with articulating features, in an example, has an articulating elongate component, associated control features and a handle. The handle of such a medical device has a hollow component, such as a cavity. Within a cavity of the handle (e.g., a housing) there are mechanisms and components that transfer movement to an articulating elongate member at a distal end of the handle. An example component to transfer movement includes a rotatable member with an arcuate track. The arcuate track can include, for example, a clamp that translates along or within the track. In an example, an articulation cable is coupled with to the clamp. In an example, the articulating cable movement, when moved by the associated articulating cable, can be transferred to the articulating elongate member. The arrangement of the clamp with the track of the rotatable member can be such that as the clamp translates within the arcuate track slack resulting from the articulating cable position is accommodated within a channel of the rotatable member.

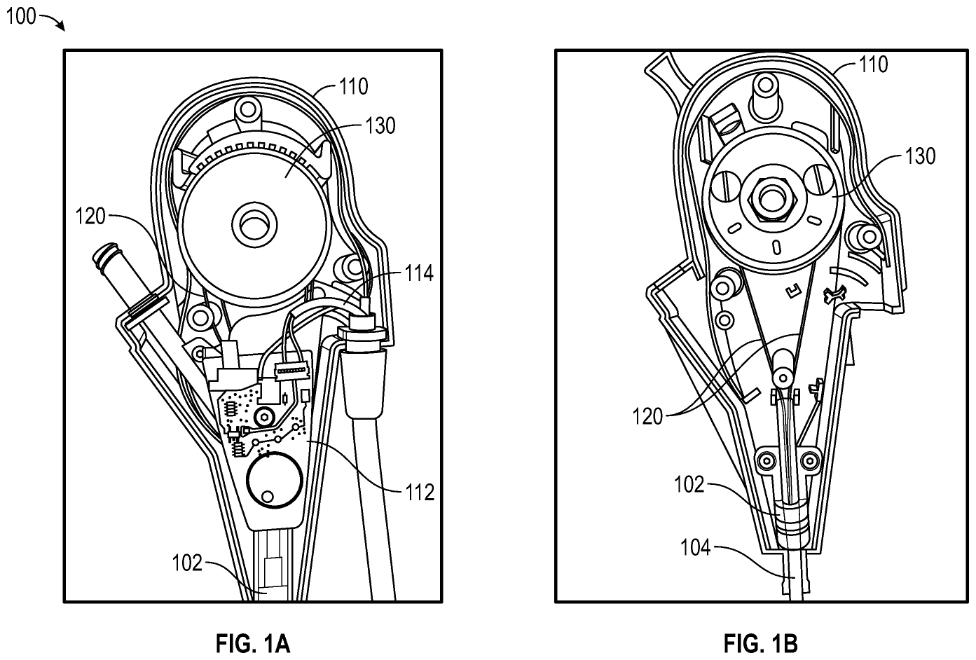

FIGS. 1A and 1B illustrate an articulating medical device 100. The articulating medical device 100 is, for example, an endoscope. In an example as shown in FIGS. 1A and 1B, the articulating medical device 100 can be an endoscope with, for example, a housing 110 which can be a handle and an endoscopic component (articulating elongate member) 102 as an insertion portion. The articulating medical device 100 controls the movement of, for example, an endoscopic component (articulating elongate member) 102 at a distal end. The articulating medical device 100 includes, for example, a housing 110 where the housing 110 contains at least electronic components 112 and associated wiring 114, at least one articulating cable 120 (hereinafter “cable”), and a rotatable member 130. The rotatable member 130 is a disc, reel, spool, pulley or any similar cam-like feature.

The rotatable member 130 is, in an example, a circular or eccentric reel. In another example, the rotatable member 130 includes a first lateral disc 133 and a second lateral disc 135. The rotatable member 130 and associated components within the housing 110 transfer movement to the articulating elongate member 102.

FIG. 1B illustrates the articulating medical device 100 without the electronic components. As illustrated in the example shown in FIG. 1B, at least one cable 120 (articulation cable) extends from the rotatable member 130 to and into the articulating elongate member 102. In an example the articulating elongate member 102 includes a tube 104 which encases, contains or encloses the cables 120. In an example, the cables 120 translate movement of the articulating elongate member 102 to move in at least two directions.

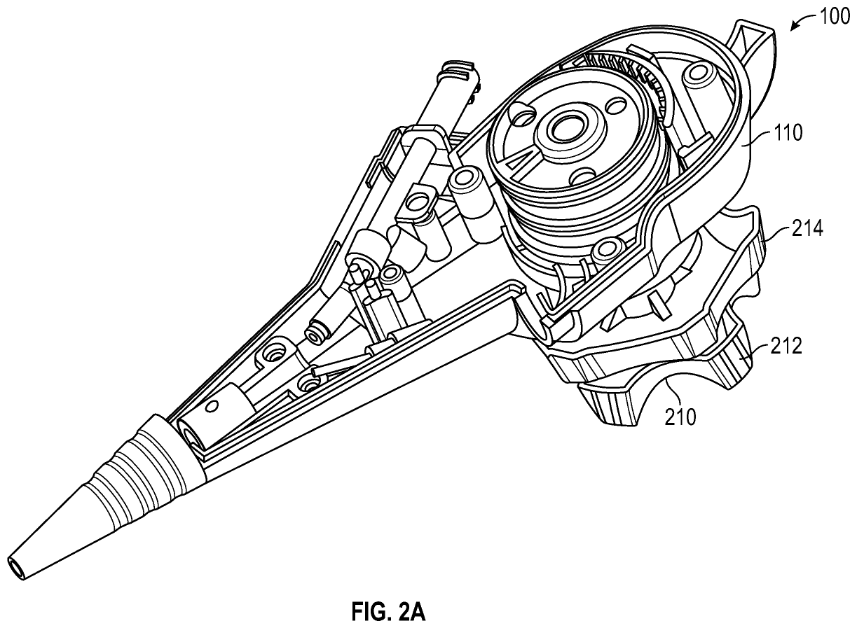

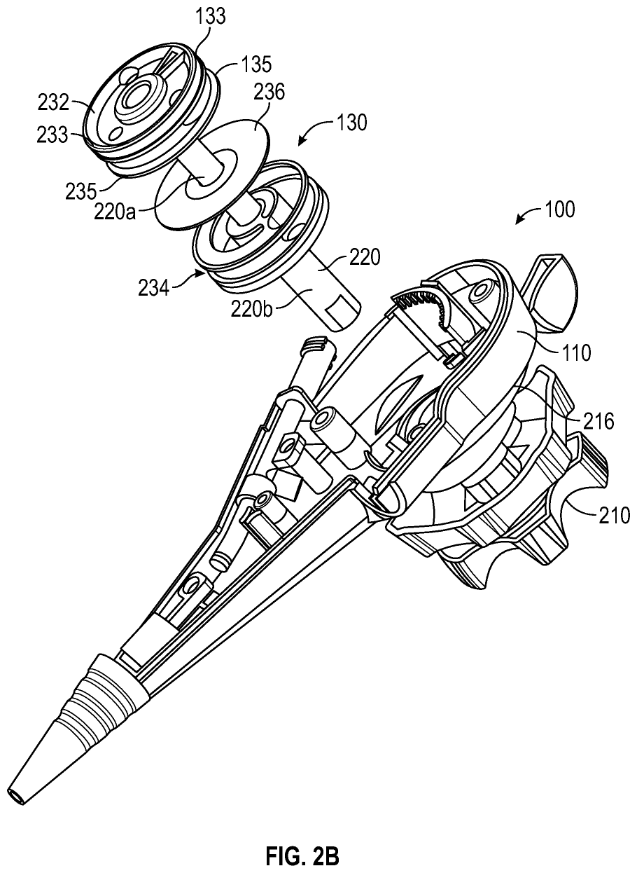

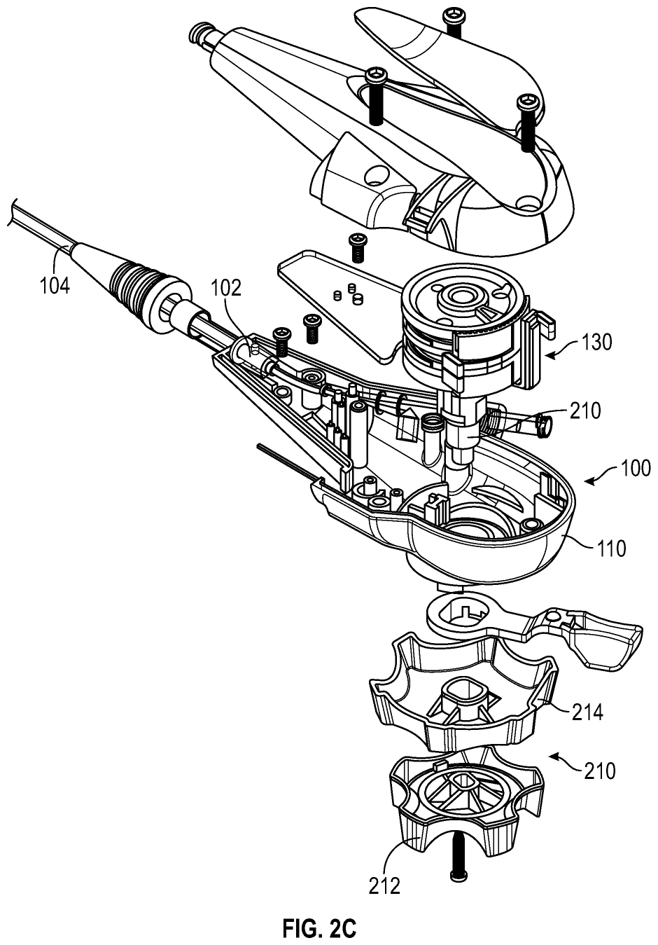

FIG. 2A illustrates a rotatable control mechanism 210 coupled to the housing 110. The rotatable control mechanism 210 is a user operated component to manipulate the rotatable member 130. The rotatable control mechanism 210 in an example has one rotatable control mechanism 212 (user manipulable control knob). The rotatable control mechanism 210 in another example has a second rotatable control mechanism 214 (user manipulable control knob). It is also contemplated to have more rotatable control mechanisms as dictated by the design.

In an example, as shown in FIG. 2B, the rotatable member 130 includes a first rotatable member 232 and a second rotatable member 234. The first rotatable member 232 rotates, for example, independently from the second rotatable member 243. In some examples, a divider or support plate 236 separates the first rotatable member 232 from the second rotatable member 234 Such that when a first rotatable member 232 and a second rotatable member 234 are used, the articulating medical device 100 can control movement in four directions.

As illustrated in FIG. 2B, the rotatable control mechanism 210 is coupled to the rotatable member 130. The rotatable member 130 has a shaft 220 extending through an outer wall 216 of the housing 110. The shaft 220 extends into the rotatable control mechanism 210. In another example, the rotatable control mechanism has a shaft or coupling which extends through the housing 110 to couple with the rotatable member. As a user operates the rotatable control mechanism 210, the rotatable member 130 moves in a reciprocal rotation. The rotatable control mechanism 210, in an example, rotates the rotatable member 130 bidirectionally about the center axis of rotation of the rotatable member 130. The first rotatable member 232 can be coupled with the associated first rotatable control mechanism 212. The first rotatable control mechanism 212 transfers motion, either horizontal motion or vertical motion from rotation of the first rotatable control mechanism 212 to the first rotatable member 232. The second rotatable control mechanism 214 transfers a different motion, either horizontal motion or vertical motion, than the first rotatable control mechanism 212, to the second rotatable member 234. In an example with multiple rotatable control mechanism, the shaft coupled to the associated rotatable member can be telescoped, inserted within another shaft of another rotatable member, or the like. In another example, the arrangement of shafts coupling the rotatable members and rotatable control mechanisms and which are extending from a rotatable control mechanism can be similarly arranged.

FIG. 2C illustrates an exploded view of an example articulating medical device 100. In the example shown in FIG. 2C, the alignment of the rotatable control mechanism 210 with the rotatable member 130 is shown to further illustrate the axis of rotation of the rotatable control mechanism 210 aligns with the axis of rotation of the rotatable member 130.

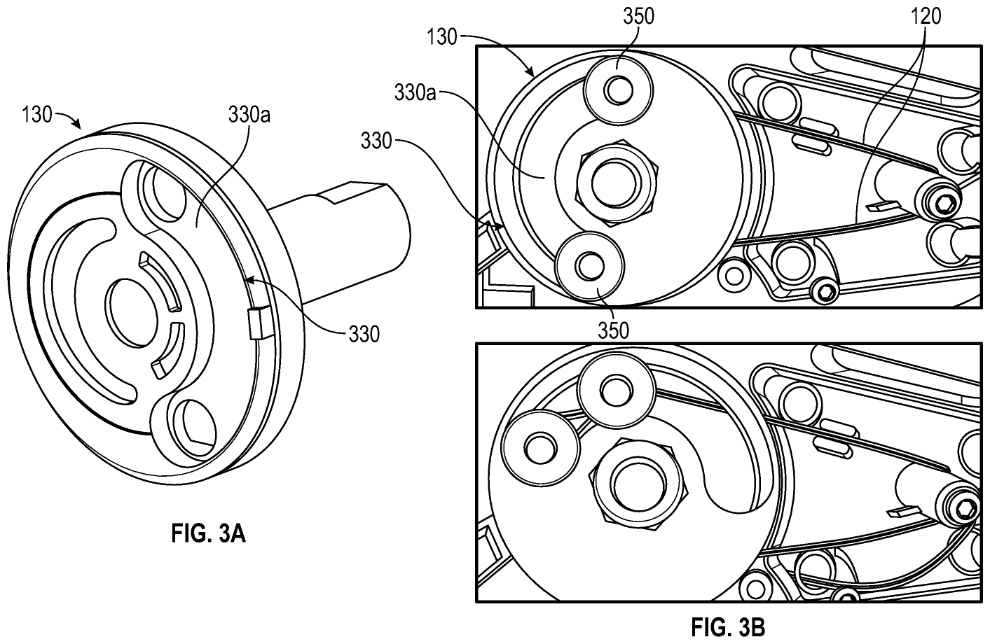

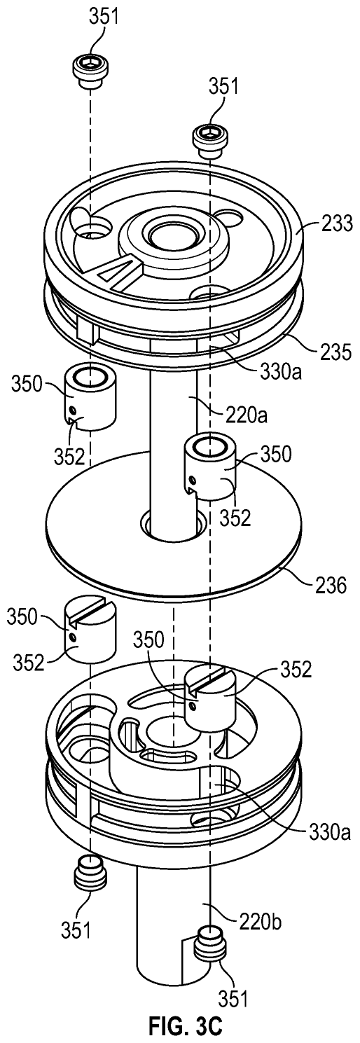

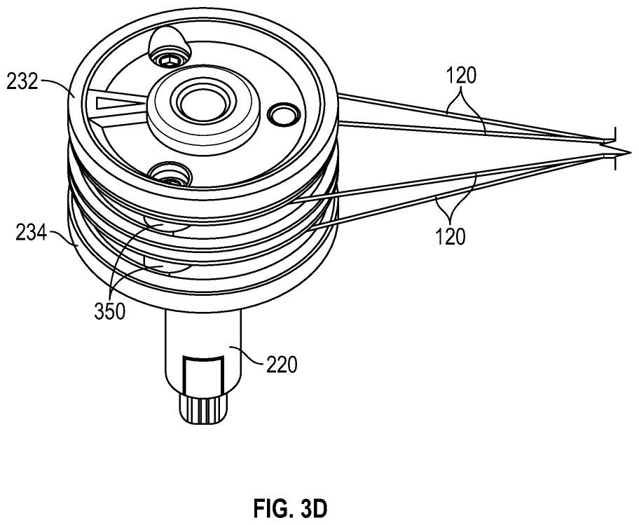

The rotatable member 130, as shown in FIGS. 3A-3D, for example, is a pulley system that rotates according to a rotation of the rotatable control mechanism 210. In an example, the rotatable member 130 (pulley system) has a track for a coupling component 350 that transfers motion to the cable 120 from rotatable member 130. In an example, the track 330 is an arcuate track. As shown in FIG. 3A, the arcuate track 330a can have openings which receive the coupling component 350. The arcuate track 330 can be a hollow track.

In an example, the arcuate track 330 extends a partial rotation about a center axis of rotation of the rotatable member 130. The rotatable member 130, including the arcuate track 330, can further include at least two coupling components 350 which secure the cables 120 to the rotatable member 130. In an example, the coupling components 350 are clamps (hereinafter clamp or clamps will refer to coupling component(s)). The clamps 350, in an example, are a barrel design (barrel clamps). In another example, the clamps 350 are wire-crimp barrel-shaped members. As illustrated in FIGS. 3c, the barrel clamp 350 (hereinafter “clamp” will refer to any fastener or coupling component to secure a cable within a rotatable member while simultaneously providing a freedom of movement), can have a flange on one end within the rotatable member 130 to allow for more stability. In another example the clamp 350 can have a base portion 351 and a body portion 352.

In another example the clamp 350 is secured within the rotatable member 130 such that the structure of the rotatable member 130 provides stability for the clamp 350. In an example where the clamp 350 is secured within the rotatable member 130, the clamps 350 are placed between a first disc 233 and a second disc 235. The first disc 233 and second disc 235 apply sufficient pressure or force to the clamps 350 to retain the clamps 350 while also providing enough freedom for the clamps 350 to travel within the space between the first disc 233 and the second disc 235 and within the arcuate track 330.

In another example the clamp 350 is disposed within a singular rotatable member 130. In such an example the clamp 350 includes a flange extending past the surface of the rotatable member. The flange secures the clamp 350 in place.

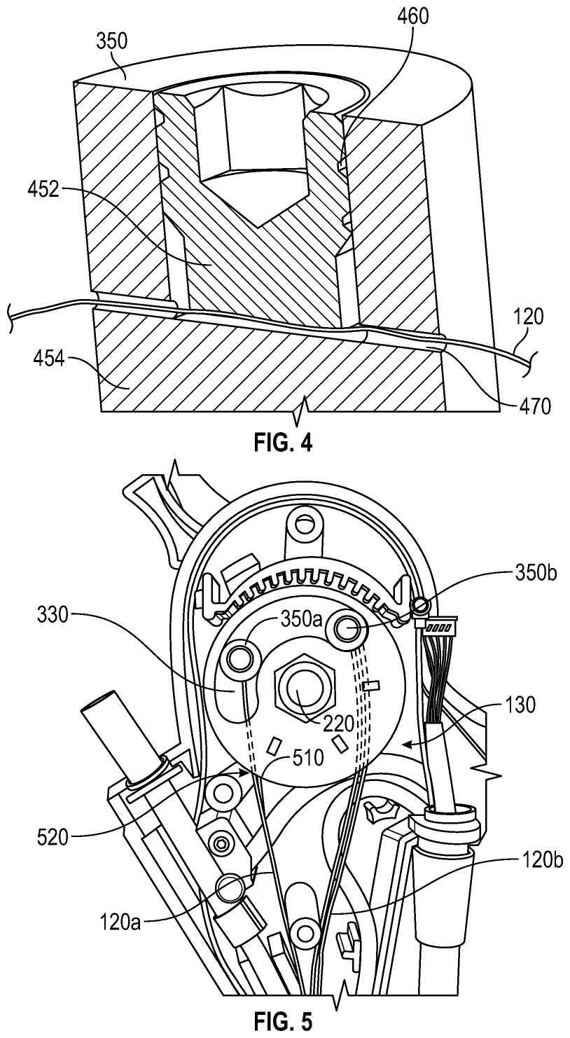

In an example shown in FIG. 3A and FIG. 3B, and as shown in FIG. 4, the clamps 350 have threads 460 for securing the cable 120 to rotatable member 130. In this example, a threaded set screw couples the cable 120 with the clamp 350. In another example, the clamp 350 has a through hole 470 which the cable 120 passes through to be tightened, secured, or otherwise coupled with the clamp 350. In an example, the cable 120 is secured by a tightened a first part 452 to a second part 454 of the clamp 350. When the first part 452 is tightened against the second part 454, a force is exerted on the cable 120 to secure the cable 120 in place within the through hole 370. In another example of a clamp with a threaded screw design, the threaded screw is external of the clamp. In another example of a clamp with a threaded screw design, the threaded screw is internal of the clamp. Other clamp designs are also contemplated to secure the cable to the clamp; the cable can be heat staked, soldered or welded to the clamp. In another example, the clamp is a two-part clamp with a first part having an upper cylindrical member and a lower cylindrical member. In this example, a pathway is formed between in the upper cylindrical member and a lower cylindrical member. The pathway allows for the cable to pass through and simultaneously captured within the clamp.

The clamps 350, for example, couple the cable 120 to the rotatable member 130, as illustrated in FIGS. 3D and 5. The clamps 350 are captured within the rotatable member 130 such that the clamps 350, for example, translate, rotate or move within the arcuate track 330. The placement of the clamps 350 within the rotatable member 130 allows, for example, the cable 120 when under slack to keep the cable 120 in an orientation that is less prone to kinking, binding or becoming entangled. The design of the rotatable member 130 in association with the clamps 350 manages the cable 120 in a slack condition and a tension condition to assist in the preventing the formation of stress points when the rotatable member 130 is in motion.

In an example shown in FIGS. 3D and 5, the clamps 350 position the cable 120 on opposing sides of the shaft 220, or center point of rotation. A first clamp 350a, for example, couples with a first cable 120a and a second clamp 350b couples with a second cable 120b. In another example, one cable is used which couples to both the first clamp 350a and the second clamp 350b. In an example, the cable 120 is positioned along a lateral periphery 510 of the rotatable member 130. The cable 120 can be positioned within a channel 520 in the lateral periphery 510, or circumference such as the circumferential periphery, of the rotatable member 130.

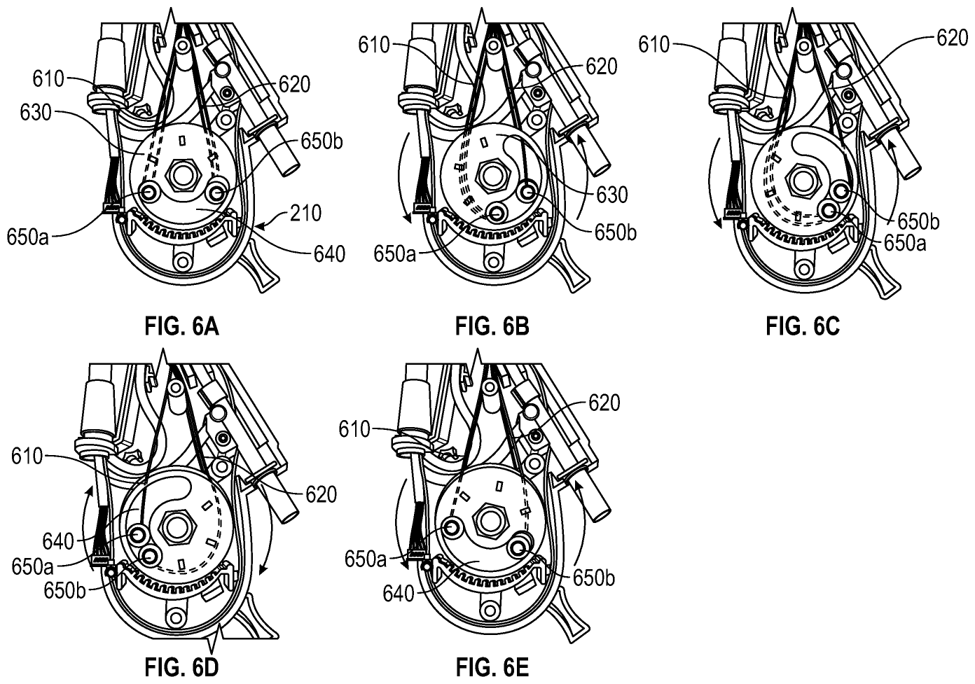

In an example shown in FIGS. 6A-6E, a method of controlling the cables in an endoscopic device is illustrated, such as when a user manipulates the rotatable control mechanism 210. The rotation of the rotatable control mechanism 210 can translate movement to the rotatable member 630. As the rotatable control mechanism 210 is rotated, the rotatable member 630 can rotate in a reciprocal movement.

As illustrated in FIG. 6A, the rotatable member is in a neutral configuration, both the first clamp 650a and the second claim 650b are approximately equally spaced within the arcuate track 640. The first clamp 650a and the second clamp 650b can be located each proximate to opposing ends of the arcuate track 640. In this configuration, a first cable 610 and a second cable 620 can be under approximately equal tension or slack.

When the rotatable member 630 rotates, as illustrated in FIG. 6B, the first clamp 650a, can be disposed, held or contained within the arcuate track 640 of the rotatable member 630. The first clamp 650a can be configured to translate or move within the arcuate track 640. The first cable 610 can be coupled with the first clamp 650a such that movement of the first clamp 650a can cause tension or slack in the first cable 610. As illustrated in FIG. 6B, when the rotatable control mechanism 210 rotates and translates movement to the rotatable member 630, the first clamp 650a can be translated with the arcuate track 640 to cause the cable 620 to be under additional tension. This tension will cause movement of the articulating member; for as the cables 610, 620 move, motion can be translated to the articulating elongate member. The second cable 620, as shown in FIG. 6C, is under slack because the rotatable member 630 has rotated in a first direction and the first cable 610 is pulled to be in tension.

The rotatable control mechanism 210 can cause the rotatable member 630 to rotate in an opposite, second direction. The rotatable member 630 can then cause the second clamp 650b to translate within the arcuate channel 640. The translation of movement can cause the second cable 620 to be pulled into tension and the first cable 610 relaxed in a slack configuration, as illustrated in FIG. 6D. In the example, the first cable 610 and the second cable 620, by being separately coupled when under tension or slack, the cables 610, 620 can avoid being tangled together.

As illustrated in the example shown in FIG. 6E, the rotatable member 630 can be then rotated back to the neutral configuration where the cables 610, 620 are under approximately equal tension or slack. The movement of the cables 610, 620 can be moved as determined by the desired position of the endoscopic device.

In some configurations, the first clamp 650a can translate and hit or come in contact with the second clamp 650b. When the first clamp 650a contacts the second clamp 650b, the second clamp 650b can rotate along with any excess cable 610. The excess cable 610 in other examples can cause rotation of the second clamp 650b. When there is excess cable, the cable can bow and therefore cause any associated clamp to rotate.

When either the first cable 610 or second cable is in a position of tension, the cable retained within the channel in the rotatable member and at least partially circumventing the rotatable member and is pulled by the clamp around the center of the rotatable member. The placement and position of the cable, retained within the channel, is controlled. The first cable attached to the first clamp is disposed on one side of the rotatable member. The second cable 620 attached to the second clamp is disposed on the opposite side of the rotatable member. Having the first and second cables disposed on opposite sides avoids entanglement between the first the second cable. The first cable and the second cable, in an example, move the articulating elongate member in a horizontal direction or a vertical direction according to the specified purpose by creating tension or slack in the cables.

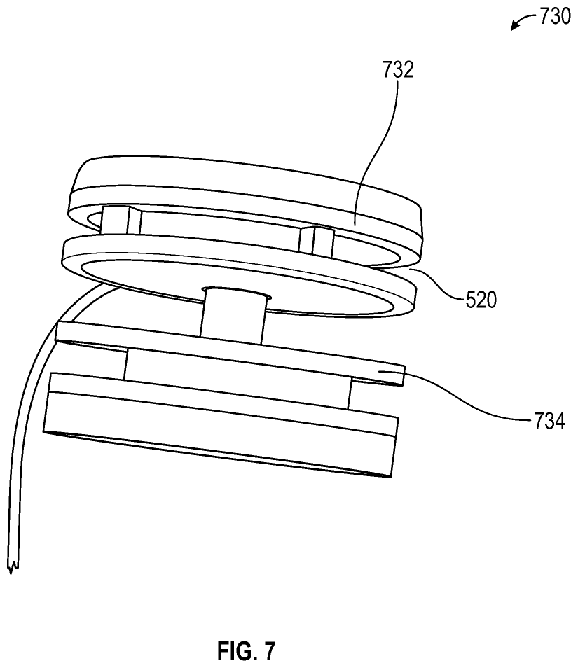

In an example as shown in FIG. 7, the rotatable member 730 has a first rotatable member 732 and a second rotatable member 734. The first rotatable member 732, for example, has a first clamp and a second clamp. The first clamp is coupled with a first cable and the second clamp is coupled with a second cable. In the example, a second rotatable member 734 is disposed parallel or nearly parallel to the first rotatable member and on the same axis of rotation as the first rotatable member 732. The second rotatable member 734 has a third clamp and a fourth clamp. The third clamp is coupled with a third cable and the fourth clamp is coupled with a fourth cable. In an example, a user manipulates the first rotatable control body. The manipulation of the first rotatable control body rotates the first rotational body. In an example, a user rotates a second rotatably control body, which then translates rotation to the second rotational body. When the first rotatable body or second rotatable body rotate, the first clamp or the second clamp are translated along the arcuate track of the associated rotatable body according to the direction of rotation of the rotatable control body. The movement of the first or second clamp (or third clamp or fourth clamp when a second rotatable body is operated) translates movement to a first cable or second cable 620 (or third cable or fourth cable when a second rotatable body is operated). The movement of the cables, control the horizontal movement of the articulating elongate member. In an example, the movement of the third clamp and the fourth clamp control the vertical movement of the articulating elongate member. Each of the first rotatable member and the second rotatable member rotates according to the movement of a respective first and second rotatable control member.

The above description includes references to the accompanying drawings, which form a part of the detailed description. The drawings show, by way of illustration, specific embodiments in which the invention can be practiced. These embodiments are also referred to herein as “aspects” or “examples.” Such aspects or example can include elements in addition to those shown or described. However, the present inventors also contemplate aspects or examples in which only those elements shown or described are provided. Moreover, the present inventors also contemplate aspects or examples using any combination or permutation of those elements shown or described (or one or more features thereof), either with respect to a particular aspects or examples (or one or more features thereof), or with respect to other Aspects (or one or more features thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

The above description is intended to be illustrative, and not restrictive. For example, the above-described aspects or examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as aspects, examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. An articulating medical device comprising:
an articulating elongate member; and
a handle, from which the articulating elongate member extends, the handle is a housing including:
at least one rotatable member including:
an arcuate track extending for a partial rotation about a center axis of rotation of the at least one rotatable member; and
at least one clamp captured and translatable within the arcuate track; and
at least one articulation cable, fastened to the at least one clamp;
wherein the at least one articulation cable extends from the rotatable member, into the articulating elongate member;
wherein the at least one articulation cable is configured to extend along a first lateral periphery of the at least one rotatable member and within a channel formed in the at least one rotatable member;
wherein the at least one clamp translates within the arcuate track to accommodate slack in the at least one articulation cable when the at least one articulation cable is not under tension.

2. The articulating medical device of claim 1 wherein the at least one rotatable member is a circular or eccentric reel.

3. The articulating medical device of claim 1 further comprising at least one user-manipulable control knob coupled with the at least one rotatable member to rotate the at least one rotatable member bidirectionally about the center axis of rotation of the at least one rotatable member.

4. The articulating medical device of claim 1 wherein the at least one articulation cable is coupled to control one of vertical movement of the articulating elongate member or horizontal movement of the articulating elongate member.

5. The articulating medical device of claim 1 wherein the at least one clamp is one of a threaded barrel-shaped member or a wire-crimp barrel-shaped member having an insertion pathway for receiving the at least one articulation cable.

6. The articulating medical device of claim 1 wherein:
the at least one clamp is a two-part clamp having an upper cylindrical member and a lower cylindrical member; and
the at least one clamp within a pathway between the upper cylindrical member and the lower cylindrical member.

7. The articulating medical device of claim 1 wherein at least a portion of the at least one clamp extends outside of the arcuate track.

8. The articulating medical device of claim 1 wherein the at least one clamp is rotatable within the arcuate track.

9. The articulating medical device of claim 7 wherein the arcuate track is defined within a center disc of the at least one rotatable member and the at least one rotatable member further includes:
a first lateral disc, toward a first side of the center disc;
a second lateral disc, toward an opposing second side of the center disc; and
the at least one clamp is captured within the arcuate track by the first lateral disc and the second lateral disc.

10. A device for controlling movement of an endoscopic component, the device comprising:
a handle having a handle body and an external first rotatable control mechanism and having an external second rotatable control mechanism;
a reel, within the handle body, wherein the reel includes:
an independently rotatable first disc including a first partial rotation arcuate track extending about a center axis of rotation of the independently rotatable first disc and a first channel along at least a portion of a circumference of the independently rotatable first disc, wherein the independently rotatable first disc is coupled with the external first rotatable control mechanism;
an independently rotatable second disc including a second partial rotation arcuate track extending about a center axis of rotation of the independently rotatable second disc and a second channel along at least a portion of the circumference of the second disc, wherein the independently rotatable second disc is coupled with the external second rotatable control mechanism;
first and second fasteners captured and translatable within the first partial rotation arcuate track; and
third and fourth fasteners captured and translatable within the second partial rotation arcuate track;
a first articulation cable coupled to the first fastener and at least partially circumventing the first independently rotatable disc within the first channel and extending from the handle including along a lateral periphery of the device;
a second articulation cable coupled to the second fastener and at least partially circumventing the independently rotatable first disc within the first channel and extending from the handle including along the lateral periphery;
a third articulation cable coupled to a third fastener and at least partially circumventing the independently rotatable second disc within the second channel and extending from the handle along the lateral periphery; and
a fourth articulation cable coupled to a fourth fastener and at least partially circumventing the independently rotatable second disc within the second channel and extending from the handle along the lateral periphery.

11. The device of claim 10 wherein the first disc includes a first center disc between first and second lateral discs and the second disc includes a second center disc between third and fourth lateral discs.

12. The device of claim 10 wherein one or more of the fasteners are barrel-shaped.

13. The device of claim 10 wherein one or more of the fasteners are rotatable within the arcuate track.

14. The device of claim 10 wherein at least one of the first, second, third or fourth cable is coupled to one of the fasteners by at least one of clamping, crimping, heat staking, soldering, or welding.

15. The device of claim 10 wherein the first rotatable control mechanism is coupled with the first disc to rotate the first disc to control movement of the endoscopic component in a horizontal direction.

16. The device of claim 10 wherein the second rotatable control mechanism is coupled with the second disc to control movement of the endoscopic component in a vertical direction.

17. A method of controlling cables within an endoscopic device, the method comprising:
transferring rotation from at least one control body on an external surface of a handle to at least one rotatable body within a cavity of the handle that includes:
a channel formed along a circumferential periphery of the at least one rotatable body;
at least one translatable and rotatable fastener disposed within an arcuate track in the at least one rotatable body; and
at least one cable, coupled to the at least one fastener, that at least partially circumvents the at least one rotatable body within the channel;
transferring rotation from the at least one rotatable body to the at least one fastener;
translating the at least one fastener along the arcuate track;
moving the at least one cable coupled to the at least one fastener so as to create one of tension or slack in a proximal end of the at least one cable; and
moving an articulating member of the endoscopic device at a distal end of the at least one cable.

18. The method of claim 17 wherein creating tension moves the articulating member one of horizontally or vertically.

19. The method of claim 17 further comprising rotating a first control body external to the handle or a second control body external to the handle.

20. The method of claim 19 further comprising:
rotating the first control body to transfer rotation to a first rotatable body; and
rotating the second control body to transfer rotation to a second rotatable body.

21. The method of claim 17 wherein rotating the at least one control body further includes:
rotating a first control body external to the handle;
rotating a second control body external to the handle;
transferring rotation from the first control body to a first rotational body;
transferring rotation from the second control body to a second rotational body;
translating at least one of a first fastener or a second fastener along the arcuate track of the first rotational body;
translating at least one of a third fastener or a fourth fastener along the arcuate track of the second rotational body;
moving at least one of a first cable coupled to the first fastener or a second cable coupled to the second fastener; and
moving at least one of a third cable coupled to the third fastener or a fourth cable coupled to the fourth fastener;
wherein moving the at least one of the first or second cable causes the endoscopic device to move horizontally and moving the at least one of the third or fourth cable causes the endoscopic device to move vertically.
